(19) United States
(12) Patent Application Publication
(10) Pub. No.: US 2008/0171907 A1
(43) Pub. Date: Jul. 17, 2008

(54) Magnetic Tissue Grasping

(75) Inventors: Gary L. Long, Cincinnati, OH (US); Ragae M. Ghabrial, Cincinnati, OH (US)

Correspondence Address: NUTTER MCCLENNEN & FISH LLP, WORLD TRADE CENTER WEST, 155 SEAPORT BOULEVARD, BOSTON, MA, 02210-2604, US

(73) Assignee: Ethicon Endo-Surgery, Inc., Cincinnati, OH (US)

(21) Appl. No.: 11/622,540

(22) Filed: Jan. 12, 2007

(51) Int. Cl. A61N 1/00; A61N 2/00

(52) U.S. Cl. 600/37; 600/12

(57) ABSTRACT

Methods and devices are provided for grasping and manipulating tissue or organs. In an exemplary embodiment, a magnetic substance is introduced into an organ, and a magnetic element is positioned adjacent to the organ to generate a magnetic field between the magnetic element and the magnetic substance, such that the magnetic element is effective to move the organ. The methods and devices are particularly useful for manipulating tissue or organs during minimally invasive surgical procedures.

### FIELD OF THE INVENTION

The present invention relates to methods and devices for performing surgical procedures, and in particular to methods and devices for manipulating tissue and organs.

### BACKGROUND OF THE INVENTION

Mechanical graspers and clamps are commonly used in a variety of medical procedures. During a laparoscopic procedure, a multitude of graspers can be introduced to provide traction and counter-traction, particularly during dissection, such as required during a cholecystectomy procedure.

In certain laparoscopic surgical procedures, mechanical graspers can be introduced into a patient's body via small incisions in the abdomen. The graspers can then assist in controlling and manipulating an organ that requires removal. However, the dissection of organs can result in complications, such as damage to surrounding organs and vessels due, for example, to the lack of visibility and difficulty in handling the organs and the graspers. In addition, the introduction of a number of instruments in a restricted body cavity further hinders the visibility and manipulation of the organ.

Likewise, a surgeon can face difficulty in manipulating an instrument, such as a mechanical grasper, for controlling an organ during endoscopic procedures conducted through a natural orifice. For example, introducing a grasper endoscopically requires that the force to operate the grasper be transmitted across a typically long and convoluted pathway from an end of the grasper that is positioned outside of the patient's body to the other end of the grasper that is positioned within the body.

Accordingly, there is a need in the art for novel methods and devices for grasping and manipulating tissue or organs in a more convenient manner, with the provision of sufficient force for control.

### SUMMARY OF THE INVENTION

The present invention generally provides methods and devices for grasping a target tissue and/or organs for enhanced ease of manipulation, particularly during minimally invasive surgical procedures. In one embodiment, an exemplary method for grasping tissue can include inserting a magnetic substance into an organ in a patient's body, positioning an instrument having a magnetic element adjacent to the organ to generate a magnetic field between the magnetic element and the magnetic substance, and manipulating the instrument to move the organ. The method can further include severing the organ from adjacent structures attached to the organ following manipulation of the organ. In one embodiment, the organ or tissue that is grasped and manipulated, is the gall bladder.

In one aspect, magnetic substance can be in the form of a fluid that includes a plurality of magnetic particles dispersed therein. The magnetic substance can alternatively be in the form of a structure such as a magnetic coil or a housing that contains magnetic material in liquid or solid form. In one embodiment, such as when the magnetic substance is in a fluid form, an additional magnetic element can be placed on or adjacent to a duct or vessel to prevent passage of the magnetic substance into other areas of the patient's body.

The instrument that includes a magnetic element can be inserted into the patient's body and placed adjacent to the organ, or it can be placed on an external portion of the patient's body, such as the abdomen, adjacent to the target organ.

### BRIEF DESCRIPTION OF THE DRAWINGS

The invention will be more fully understood from the following detailed description taken in conjunction with the accompanying drawings, in which:

FIG. 1 is a perspective view showing one embodiment in which a magnetic substance is disposed within an organ;

FIG. 2 is a perspective view of another embodiment in which another form of a magnetic substance is disposed within an organ;

FIG. 3 is a perspective view of an instrument having a magnetic element formed thereon that is useful in practicing the invention;

FIG. 4 is a perspective view illustrating an aspect of the invention in which another instrument having a magnetic element is positioned external to an organ;

FIG. 5 illustrates one embodiment of a method for grasping tissue ; and

FIG. 6 illustrates another embodiment of a method for grasping tissue.

### DETAILED DESCRIPTION OF THE INVENTION

Certain exemplary embodiments will now be described to provide an overall understanding of the principles of the structure, function, manufacture, and use of the devices and methods disclosed herein. One or more examples of these embodiments are illustrated in the accompanying drawings. Those skilled in the art will understand that the devices and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

The present invention generally provides methods and devices for holding and manipulating tissue, and in particular for grasping a target tissue or organ with the assistance of a magnetic field. The term “tissue” is used herein in its broad sense and includes any tissue structure within the body, including organs.

According to methods described herein, a magnetic substance can be introduced into a tissue, such as an organ tissue. By positioning an instrument having another magnetic element adjacent to the target tissue, a magnetic force is generated such that the target tissue, e.g., an organ, is effectively grasped by the instrument and it is thus capable of being manipulated. Grasping a tissue or organ magnetically is advantageous in that it enables easy and effective manipulation of tissue and/or organs, particularly when there is a need to control tissue or organs during minimally invasive surgical procedures. In addition, the use of a magnetic force to grasp tissue or an organ provides such benefits as improved traction, particularly during dissection of a tissue or an organ.

While the devices and methods disclosed herein can be used in conventional, open surgical procedures, they are particularly useful in minimally invasive surgical procedures, such as laparoscopic and endoscopic procedures. The principles described herein can be applicable to the particular types of tools described herein, and to a variety of other surgical tools having similar functions. In addition, the tools can be used alone in a surgical procedure, or they can be used in conjunction with other devices, such as endoscopes, that facilitate minimally invasive surgical procedures.

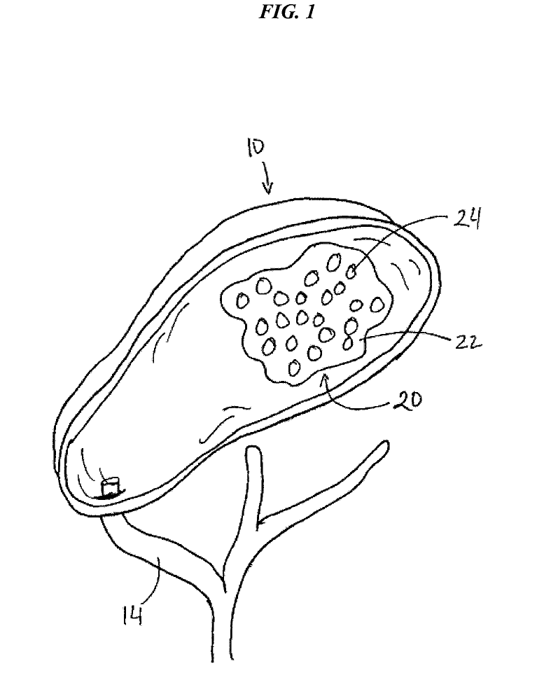

FIG. 1 illustrates one example of a magnetic substance 20 that can be introduced into tissue or an organ 10. In one embodiment, the magnetic substance 20 can be in the form of a magneto-rheological fluid in which a biocompatible fluid medium 22 has a plurality of magnetic particles 24 dispersed therein. The magnetic substance 20 can be introduced into a cavity within the organ or it can be introduced into tissue forming the organ.

The fluid medium 22 can be virtually any biocompatible material such as a liquid or a gel. By way of non-limiting example, a fluid material can include MRF-132DG (Lord Corp., Cary, N.C.). MRF-132DG is a hydrocarbon based fluid magneto-rheological fluid, formed of a suspension of micron-sized, magnetizable particles. The fluid medium 22 can also include a wide viscosity range. In one example, the fluid can have a viscosity such that the fluid can be pass through an injection needle.

Similarly, the magnetic particles 24 can be any biocompatible magnetic materials having sufficient magnetic force to enable the tissue and/or organ within which they are disposed to be manipulated with an instrument having another magnetic material associated therewith. Exemplary materials from which the magnetic particles 24 can be formed include any steel material with a sufficient iron content to become magnetized.

In general, the magnetic particles 24 can have varying sizes and configurations. In one embodiment, the magnetic particles 24 can have a diameter such that when introduced into the organ 10, the magnetic particles 24 are unable to migrate from the tissue and/or organ, for example by passing through an artery 14 coupled to the organ 10. This avoids any passage of the magnetic particles 24 into the circulatory system. By way of example, the magnetic particles 24 can also have a configuration that enables the particles to be introduced into a tissue or organ by a variety of techniques. For example, the magnetic substance can be introduced through a device, such as a cannula, by an endoscopic or percutaneous technique.

One skilled in the art will further appreciate that the concentration of the magnetic particles can vary depending upon that specific surgical procedure that is utilized. In general, the concentration of magnetic particles 24 should be sufficient to generate a magnetic force that enables the tissue and/or organ to be manipulated with an instrument having another magnetic material associated therewith. In one embodiment the concentration of the magnetic particles can be in the range of about 50-80%.

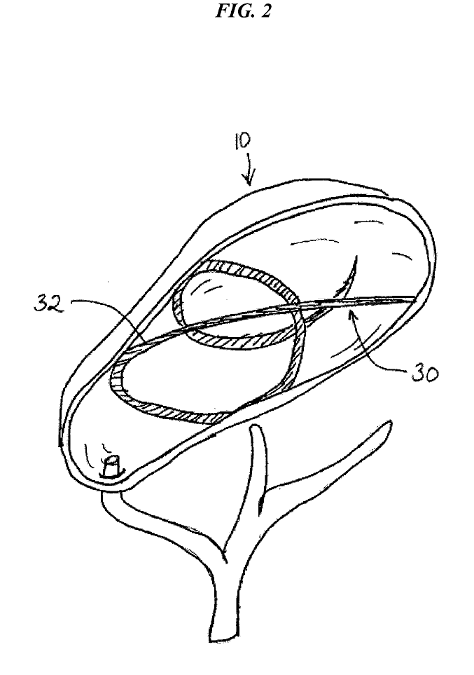

FIG. 2 illustrates another embodiment of a magnetic substance that can be introduced into a tissue and/or organ. In this embodiment the magnetic substance 30 is in the form of a structure that can be disposed on or within tissue and/or an organ. The magnetic substance 30 itself can be a magnetic structure or it can include a housing 32 that can contain magnetic particles (not shown), which may or may not be in a fluid medium. In an alternative embodiment, the magnetic substance 30 can be formed of a solid wire having a sufficient amount of iron contained therein to become magnetized. The magnetic materials that can be incorporated within or used to form magnetic substance 30 are of the type described above with respect to FIG. 1. The magnetic force that these materials possess may likewise be as described above.

The housing 32 can have a design and properties that enable it to be delivered to a target site through a minimally invasive technique such as a laparoscopic or an endoscopic technique. In general, the housing 32 can be rigid or flexible. In an exemplary embodiment, at least a portion of the housing 32 is sufficiently flexible to allow the housing 32 to be inserted endoscopically through a natural orifice and through a tortuous body lumen. In one embodiment, the housing 32 can be formed from an elongate tubular member. In another exemplary embodiment, the housing 32 can have a coiled configuration, as shown in FIG. 2. Alternatively, the housing 32 can be formed from any structure that is capable of engaging tissue. The housing 32 described herein can be formed from a variety materials, including, by way of non-limiting example, metals such as aluminum, stainless steel or titanium, shape-memory materials, polymers, or various other biocompatible materials. To ensure that the housing 32 can be introduced into a tissue or organ, the housing can have a diameter sufficiently narrow to allow the housing 32 to be injected into the tissue or organ.

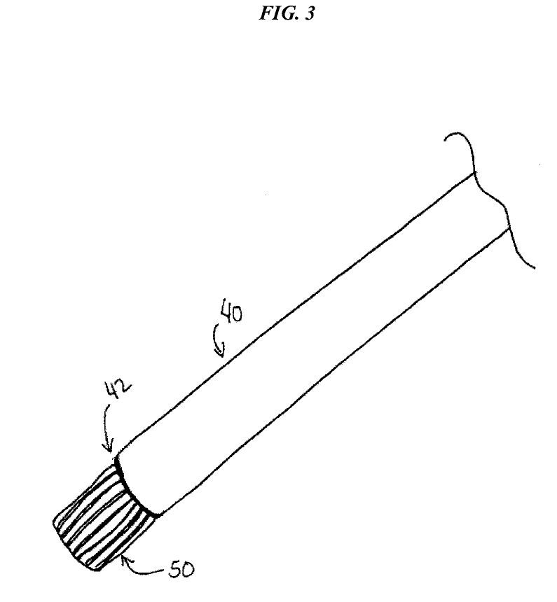

FIG. 3 illustrates one example of an instrument 39 having a magnetic element that can be used in connection with the present invention. As shown, in this embodiment the magnetic element 50 can be positioned at the distal end 42 of an elongate member 40. The elongate tubular member 40 can have a variety of configurations, but in one embodiment it is an elongate, tubular member configured such that the proximal end (not shown) can remain outside a patient's body, while the distal end 42 is positioned within the body. The particular configuration of the elongate tubular member 40 can vary depending on the type of procedure being formed. For example, the elongate tubular member 40 can be rigid or flexible, and it can vary in shape, size and length. The magnetic element 50 can be mated to the elongate tubular member 42 in a variety of ways. For example, the magnetic element 50 can be integrally formed with the distal end 42 of the elongate tubular member 50, or it can be attached thereto, removably or permanently, by way of a variety of known mating techniques.

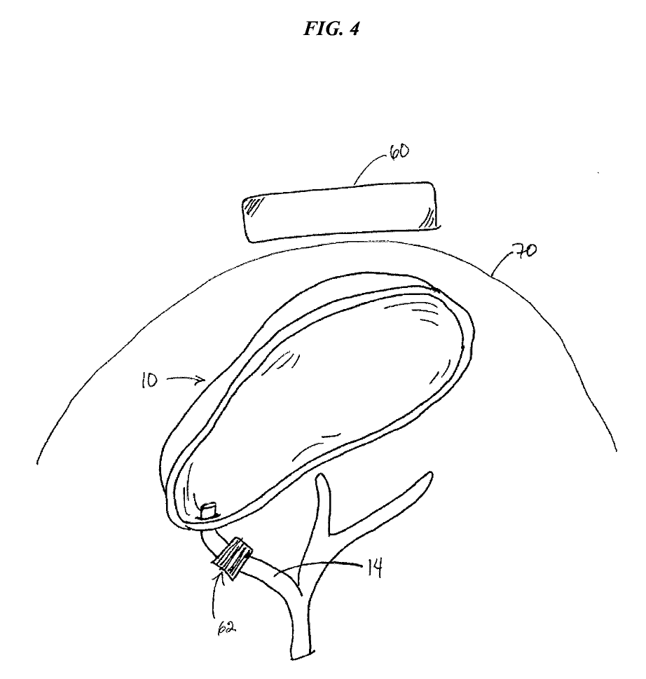

FIG. 4 illustrates another embodiment of an instrument having a magnetic element. In this embodiment, the instrument can be a magnetic element that can be formed from an independent magnetic component. The shape and size of the magnetic element can be of any variety, for example, small, large, round, oval square, thick or thin. FIG. 4 further illustrates one embodiment of an independent magnetic element in use. As shown, a first magnetic element 60 can be positioned at a location external to the patient's body such that it is located external but adjacent to the target organ 10. In the exemplary embodiment, the first magnetic element 60 is positioned on an external surface of an abdominal wall 70. In addition, a second magnetic element 62 can optionally be internally placed adjacent to a duct or an artery 14, which extends from the organ 10. As explained below, the second magnetic element 62 can be useful to prevent the passage of magnetic material, such as magnetic particles in a fluid medium, from the target organ to other areas of the patient's body.

In use, the magnetic element 60 (as well as magnetic element 62 if used) serves as the counter component to the magnetic substance that will be delivered to the organ, resulting in the attractive force of the magnetic fields. The magnetic energy between a magnetic substance and a magnetic element enables a tissue or organ to be grasped magnetically with the assistance of the magnetic element once a magnetic substance has been introduced into a target organ or tissue.

One skilled in the art will appreciate that the magnetic element can be formed from any component that will create a magnetic attraction between itself and a magnetic substance. In one exemplary embodiment, the magnetic element can be an electromagnet. In another embodiment, the magnetic element can be a permanent magnet.

One skilled in the art will appreciate that it may be necessary or desirable to provide devices and methods for containing the magnetic substance within an organ or a tissue once it has been introduced. That is, the magnetic substance should be prevented from migrating out of the target organ or tissue through an opening or conduit that is either natural or surgically created. In one exemplary embodiment, a sealing mechanism (not shown) can be used to block a surgically created opening through which the magnetic substance was introduced into the tissue or organ. Such a sealing mechanism can have any configuration or size and be formed of any material which is effective to seal an opening in tissue. The sealing mechanism ensures that the magnetic particles in a magnetic substance, such as a magnetic fluid, are prevented from diffusing out of the organ or tissue through a surgically created opening.

Unwanted diffusion of the magnetic substance from a housing that contains a magnetic substance such as described with respect to FIG. 2, can be prevented by the use of a sealing mechanism disposed in the housing to prevent the magnetic particles from leaking out of the interior of the housing. Such a sealing mechanism can be useful to ensure that none of the magnetic material can diffuse into the surrounding tissue or organs once the housing containing the magnetic substance has been introduced into the organ or tissue.

It may also be desirable to prevent the passage of the magnetic substance from the target tissue or organ through a natural opening or conduit. FIG. 4 illustrates an embodiment in which a magnetic element, such as a ring or coil 62, is placed around a duct or vessel to prevent passage of the magnetic substance into other areas of the patient's body such as the circulatory system.

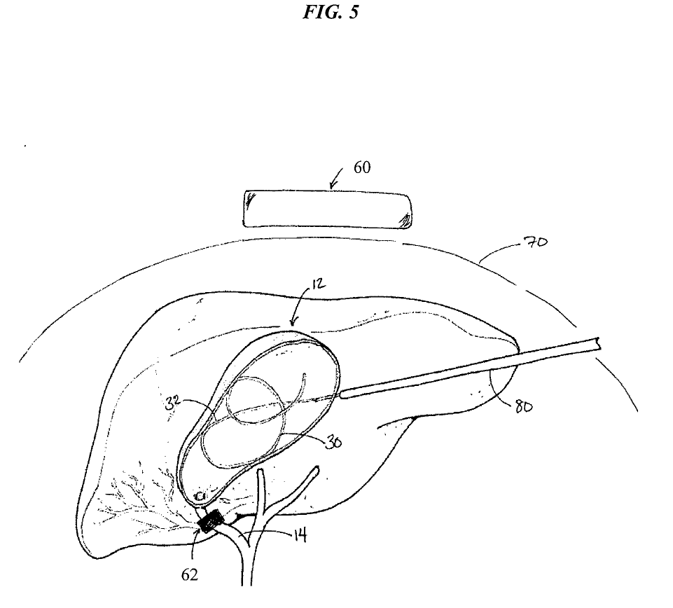

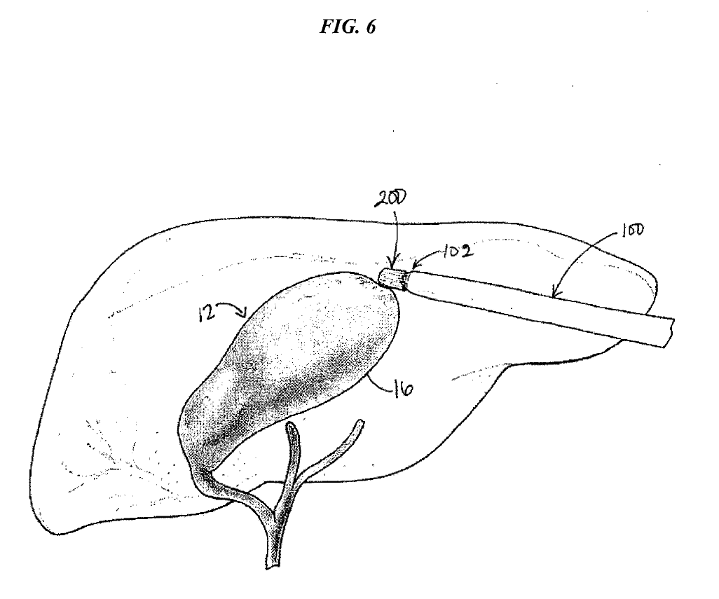

Exemplary methods for magnetically grasping and manipulating a tissue or organ are further described with reference to FIGS. 5 and 6. After preparing a patient for surgery, an organ, such as the gall bladder 12, can be accessed and prepared using techniques known in the art. Following the preparation of the gall bladder 12, at least a portion of the bile contained therein can be drained or removed from the gall bladder 12. As shown in FIG. 5, a sheath or catheter 80, which can be delivered laparoscopically or endoscopically, can then be positioned adjacent a surface of the gall bladder, and a magnetic substance 30 can be introduced into the gall bladder. FIG. 5 illustrates the magnetic substance 30 to be in the form of a coiled housing 32 that contains a magnetic particles. However, one skilled in the art will understand that the magnetic substance can be in any form or configuration, as described above. Once the magnetic substance 30 is implanted, the point of entry into the gall bladder 12 can be sealed using any suitable sealing mechanism, including one positioned on a proximal end of the housing 32.

Following the insertion of the magnetic substance within the gall bladder 12, a magnetic element can be employed to grasp and manipulate the gall bladder 12. In one exemplary method, as illustrated in FIG. 5, a magnetic element 60 is externally positioned in the vicinity of the gall bladder 12, such as adjacent an exterior surface of the abdominal wall 70 of a patient's body. As further illustrated in FIG. 5, a second magnetic element 62 can be positioned adjacent to or around an artery 14 leading to the gall bladder 12 to prevent the passage of magnetic material into the patient's circulatory system.

In another embodiment, as illustrated in FIG. 6, organ manipulation can be effected using a catheter 100 having a magnetic component 200 coupled to a distal end 102. The catheter 100 can be delivered to a position adjacent an outer wall 16 of the gall bladder 12 using laparoscopic or endoscopic techniques, and thereafter the organ can be manipulated.

Once the magnetic element is positioned through a technique as described above, a magnetic field is generated between the magnetic element and the magnetic substance, resulting in the magnetic substance being drawn toward the magnetic element. The magnetic substance thus becomes concentrated along an inner wall of the gall bladder such that it is positioned in the vicinity of the magnetic element. The magnetic attraction, as noted above, can be used to assist in manipulating the organ so that it can be severed and removed.

The magnetic attraction between the magnetic element and the magnetic substance renders the magnetic element effective to grasp and control the gall bladder as desired. The grasping force can be varied such that if the magnetic element is applied directly to a surface wall of the gall bladder, as shown in FIG. 6, the grasping force can either be a hard grasp or a soft grasp. A soft grasp, for example, can be one in which the magnetic element is slidable along a surface of the gall bladder while maintaining sufficient force to manipulate the gall bladder. In one embodiment, a soft grasp can be effected by the magnetic component including a circular cross-section such that when the magnetic component is positioned adjacent an outer wall of the gall bladder, the magnetic component can be “rolled” along the surface of the gall bladder, while still maintaining an attractive force with a magnetic substance. A hard grasp is one in which the magnetic attraction is such that movement of the magnetic element along the organ surface will result in movement of the organ as well. In an exemplary embodiment, a hard grasp can by effected by the magnetic component including a flat surface, such that when the magnetic component is positioned on a surface of the gall bladder, the gall bladder will be much less likely to move relative to magnetic component. One skilled in the art will appreciate that the force by which a magnetic component grasps a tissue or an organ surface will depend on the magnetic concentration of the magnetic substance as well as the surface area of the magnetic component. Accordingly, properties can be tailored to achieve a result necessary or desirable for a given application.

The method described above provides many advantages. For example, the grasping of the gall bladder enables the gall bladder to lifted away from the liver, such that a cholecystectomy can be more readily and simply performed by minimally invasive surgical techniques. While the above method has been described in terms of manipulation of the gall bladder, one skilled in the art will appreciate that the methods and devices described herein are equally applicable to the manipulation of other organs or tissue in a variety of minimally invasive surgical procedures.

One skilled in the art will appreciate further features and advantages of the invention based on the above-described embodiments. Accordingly, the invention is not to be limited by what has been particularly shown and described, except as indicated by the appended claims. All publications and references cited herein are expressly incorporated herein by reference in their entirety.

What is claimed is:

1. A method for grasping tissue, comprising:
inserting a magnetic substance into a target tissue in a patient's body;
positioning an instrument having a magnetic element adjacent to the target tissue to generate a magnetic field between the magnetic element and the magnetic substance; and
manipulating the magnetic element to move the target tissue.

2. The method of claim 1, wherein the magnetic substance comprises a magnetic fluid that is injected into the target tissue.

3. The method of claim 2, wherein the magnetic fluid comprises a plurality of magnetic particles dispersed in a fluid medium, and wherein the magnetic particles have a size that prevents drainage of the particles from the organ into an artery coupled to the target tissue.

4. The method of claim 1, wherein the magnetic substance comprises a magnetic coil that is inserted into the target tissue.

5. The method of claim 1, wherein the magnetic element is coupled to a distal end of a catheter disposed through a body lumen and positioned adjacent to the target tissue.

6. The method of claim 1, wherein the instrument having a magnetic element is positioned external to the patient's body.

7. The method of claim 6, wherein the instrument having a magnetic element is positioned adjacent to an abdominal wall of the patient's body.

8. The method of claim 1, wherein the target tissue is an organ.

9. The method of claim 8, wherein the organ comprises a gall bladder.

10. The method of claim 1, further comprising positioning a second instrument having a magnetic element adjacent to an artery coupled to the target tissue to prevent draining of the magnetic fluid from the organ.

11. The method of claim 8, further comprising, following manipulating the organ, severing the organ from adjacent structures attached to the organ.

12. The method of claim 1, wherein the magnetic field is electrically generated.

13. A method for grasping tissue, comprising:
delivering a magnetic material into a tissue wall;
positioning an instrument having a magnetic component adjacent to the tissue wall;
generating a magnetic attraction between the magnetic material and the instrument such that a force is created whereby the instrument is capable of grasping the tissue wall.

14. The method of claim 13, further comprising moving the instrument to thereby move the tissue wall.

15. The method of claim 13, wherein delivering a magnetic material into the tissue wall comprises injecting a fluid containing a plurality of magnetic particles into the tissue wall.

16. The method of claim 13, wherein delivering a magnetic material into the tissue wall comprises inserting a housing containing a plurality of magnetic particles into the tissue wall.

17. The method of claim 13, wherein positioning the instrument having a magnetic component comprises inserting a catheter through a body lumen to position a distal end of the catheter having the magnetic component located thereon adjacent to the tissue wall.

18. The method of claim 17, wherein instrument having a magnetic component grasps the tissue wall with a force sufficient to enable the catheter to manipulate the tissue wall.

19. The method of claim 13, wherein the instrument having a magnetic component is positioned on an external tissue surface.

20. The method of claim 13, wherein generating a magnetic attraction between the magnetic material and the instrument having a magnetic component comprises activating the magnetic component to generate a magnetic field.
